(19) United States
(12) Patent Application Publication
(10) Pub. No.: US 2022/0331270 A1
(43) Pub. Date: Oct. 20, 2022

(54) LACTIC ACID VAGINAL FILM AND IT'S PROCESS

(71) Applicant: Azista Industries Pvt Ltd, Hyderabad (IN)

(72) Inventors: Srinivas Reddy Male, Hyderabad (IN); Shantaram Laxman Pawar, Hyderabad (IN); Lakshman Rao Sakare, Hyderabad (IN); Jaipal Arikotla, Hyderabad (IN)

(21) Appl. No.: 17/762,137

(22) PCT Filed: Sep. 23, 2020
(86) PCT No.: PCT/IB2020/058858

§ 371 Date: Mar. 21, 2022

(30) Foreign Application Priority Data
Sep. 24, 2019 (IN) 201941038558

(51) Int. Cl. A61K 31/19; A61K 9/00; A61K 9/70; C08J 7/06

(52) U.S. Cl. CPC A61K 31/19; A61K 9/0034; A61K 9/7007; C08J 7/065

(57) ABSTRACT

The present invention relates to a composition of vaginal film comprising Lactic acid as active ingredient, optionally prebiotics and pharmaceutically acceptable excipients. The present invention also relates to a composition of organic acid vaginal film comprising Lactic acid as active ingredient, optionally fructooligosaccharides as prebiotic, film forming agents, plasticizers, neutralizing agent, lubricants as pharmaceutically acceptable excipients and preservatives, disintegrating agents, anti-foaming agent, antimicrobial agent, coloring agents, flavouring agents as optional excipients. The present invention also relates to process for the preparation of Lactic acid vaginal film comprising the steps of dissolving/melting, mixing, coating/coating & drying and cutting.

### FIELD OF INVENTION

The present invention relates to a composition of vaginal film comprising Lactic acid as active ingredient, optionally prebiotics and pharmaceutically acceptable excipients.

The present invention also relates to a composition of organic acid vaginal film comprising Lactic acid as active ingredient, optionally fructooligosaccharides as prebiotic, film forming agents, plasticizers, neutralizing agent, lubricants as pharmaceutically acceptable excipients and preservatives, disintegrating agents, anti-foaming agent, antimicrobial agent, coloring agents, flavouring agents as optional excipients for prevention of urogenital tract infections.

The present invention also relates to a composition of organic acid vaginal film comprising Lactic acid as active ingredient, fructooligosaccharides as prebiotics, film forming agents, plasticizers, neutralizing agent, lubricants, anti-foaming agent, antimicrobial agent, preservatives, disintegrating agents, coloring agents, flavouring agents as pharmaceutically acceptable excipients for prevention of urogenital tract infections.

The present invention also relates to process for the preparation of Lactic acid vaginal film comprising the steps of dissolving/melting, mixing, coating/coating & drying and cutting.

### BACKGROUND OF INVENTION

Urogenital infections, including urinary tract infections (UTI), bacterial vaginosis (BV) and yeast vaginitis are common disorders in women all around the world. More than 75% of women will have at least one vaginal infection in their lives, and 50% of these women will have a recurrence of the infection. Vaginal infections are currently treated mainly with antibiotics. Antibiotics, however, also kills useful bacteria present in the vaginal environment, such as Lactobacilli, resulting in a pH increase in the vaginal environment and increasing risk of recurrence of the bacterial vaginosis or the development of a different vaginal infection, such as a yeast infection.

Most commonly, this presents clinically with increased vaginal discharge that has a fish-like odor. The discharge itself is typically thin and either grey or white. After being diagnosed with bacterial vaginosis, women have an increased risk of acquiring other sexually transmitted infections (STI) and pregnant women have an increased risk of early delivery.

Lactic acid allows the maintenance of an acidic pH around 3.5-4.5 and it is not favourable for the growth of fungi, protozoa and other unwanted bacteria, which generally need a pH greater than 6.0. On the contrary, lactobacilli are acidophilic, i.e. they feel comfortable in an acidic environment, which enables their maximal proliferation.

Valore, E. V., et al., American Journal of Obstetrics and Gynecology, 2002, 187(3), 561-568 discloses that Lactic acid and low pH of the vaginal fluid create a selective antimicrobial barrier against harmful pathogens that can cause vaginal infections such as bacterial vaginosis.

U.S. Pat. No. 7,897,080 B2 discloses film products and methods of their preparation that demonstrate a non-self-aggregating uniform heterogeneity. Desirably, the films disintegrate in water and may be formed by a controlled drying process, or other process that maintains the required uniformity of the film. The films contain a polymer component, which includes polyethylene oxide optionally blended with hydrophilic cellulosic polymers. Desirably, the films also contain a pharmaceutical and/or cosmetic active agent with no more than a 10% variance of the active agent pharmaceutical and/or cosmetic active agent per unit area of the film. It also discloses that films may be used for the administration of an active to any of several body surfaces, especially those including mucous membranes, such as vaginal administration. However, this patent does not disclose Lactic acid as active ingredient.

WO Publication No. 03/000224 A1 discloses pharmaceutical composition for vaginal administration comprising a gel based on chitosan and Lactic acid. It also discloses topical application at vaginal level, for the treatment of various types of bacterial vaginosis and for the restoring (recolonising) of the physiological flora of lactobacilli having good biodegradability and biocompatibility with the vaginal environment, with a pH that falls within the range of physiological values of the vaginal cavity, having improved muco-adhesive properties and avoiding undesired contraceptive effects.

WO Publication No. 2004/105822 A1 discloses film-shaped polymer matrixes comprising Lactic acid producing bacteria in a film-shaped matrix consisting of polymer(s) that is non-toxic and non-irritant to a user's skin and mucous membranes. It also discloses process for producing such a film-shaped polymer matrix and products containing it. The film-shaped polymer matrixes protect bacterial cells from moisture thereby increasing bacterial survival during transport and storage.

WO Publication No. 2005/013906 A2 discloses pH-responsive film comprising: (a) a biocompatible, hydrophilic polymer that is positively charged at a first pH and in electronically neutral form at a higher pH; and (b) an alkylene oxide polymer or copolymer. Film comprises different compositions of chitosan lactate, pluronic 108, HPMC 50, D, L-Lactic acid, citric acid and glycerin. pH responsive films can be used for contraception, treatment and/or prevention of viral infections, treatment of vaginal infections, relief of vaginal itch, vaginal cleansing, and enhancement of vaginal lubrication.

WO Publication No. 2009/043588 A2 discloses antibacterial films that may regulate pH and control bacterial growth in the oral or vaginal environment and methods of use thereof. The films may include Lactic acid as pH adjusting ingredient, eucalyptus oil, menthol, peppermint oil, crisp mint oil, spearmint oil, Pelargonium sidoides root extract as antimicrobial essential oil and polyvinyl alcohol, polyethylene glycol, rice starch as polymer. The polymer may include polyvinyl alcohol, polyethylene glycol and rice starch. The vaginal films and methods of use may treat infections in the vagina.

Sridevi et al., Journal of Pharmacy Research, 2014, 8(3), 321-330 discloses that the main advantage of prebiotic oligosaccharides is that they are natural functional ingredients. Prebiotics are generally defined as non-digestible polysaccharides and oligosaccharides (NDO), which promote the growth of beneficial Lactic acid bacteria in the colon and exert antagonism to Salmonella sp. or Escherichia coli, limiting their proliferation.

J. Haya et al., Open Journal of Obstetrics and Gynecology, 2014, 4, 787-799 discloses that in normal conditions, a large number of saprophytes can be found in the vagina, being the most important acidophilic bacteria which produce Lactic acid. Lactic acid keeps the vagina within an acidic pH between 3.5 and 4.5, which is the ideal environment for acidophilus bacilli to find a suitable medium to metabolize. This makes it possible for these bacteria to produce large amounts of Lactic acid, which maintains an acidic pH, and creates a virtuous circle. The origin of vaginitis and vaginosis lies in the alteration of the number of acidophilus bacilli and/or the alkalization of the vaginal pH, allowing the proliferation of common vaginal saprophytes, which become pathogenic and produce symptoms. The treatment of vaginitis and vaginosis should deal not only with the reduction in the number of causing-symptoms pathogens, as done traditionally, but also with the primary objective to recover the vaginal pH and restore the virtuous circle that maintains a healthy vagina.

WO Publication No. 2016/020861 A2 discloses film or sponge compositions, and to a method for producing the same for the prevention and treatment of urogenital infections, in particular vaginal infections. A water-based composition for producing a film or sponge for use in the treatment of urogenital infections comprises: 0.4-25% (m/m) plasticiser, 0.4-20% (m/m) of at least one mucoadhesive polymer, at least one probiotic and/or prebiotic, at least one active compound in pharmacologically effective amounts. It also discloses Lactic acid producing bacteria, particularly Lactobacillus spp. as probiotics and Lactic acid as prebiotic.

WO Publication No. 2016/053308 A1 discloses prebiotic compositions and formulations comprising an α-hydroxy acid and salts thereof, such as Lactic, glycolic, citric, tartaric or malic acid and a prebiotic agent, such as inulin, fructo-oligosaccharide (FOS), lactulose, galacto-oligosaccharide (GOS), raffinose, stachyo se, isomalto-oligosaccharide, and xylo-oligosaccharide. It also discloses that prebiotic formulation is in the form of liquid, solution, paste or gel.

WO Publication No. 2017/196006 A1 discloses combination of salt, sugar and Lactic acid bacteria as an active ingredient to treat or prevent vaginosis.

WO Publication No. 2019/006122 A1 discloses hot melt extrusion as a process for forming vaginal drug delivery films. The method involves extruding a composition comprising one or more active pharmaceutical ingredients and one or more polymer carriers at an elevated temperature through a die to thereby provide the film. Method of preparing a vaginal drug delivery film, comprising: extruding through a die a composition comprising one or more active pharmaceutical ingredients, from 35% to 60% by weight of a high molecular weight polyethylene oxide carrier having a molecule weight of from 100,000 to 700,000 Da, and from 15% to 30% by weight of a medium molecular weight polyethylene oxide carrier having a molecule weight of from 3000 to 8000 Da, to thereby provide the film. It also discloses that the Lactic acid produced by Lactobacillus, a by-product of the metabolism of glycogen released by vaginal epithelial cells, allows the vaginal fluid to remain acidic (healthy vaginal pH 3.5-4.7). The Lactic acid and low pH of the vaginal fluid create a selective antimicrobial barrier against harmful pathogens that can cause vaginal infections such as bacterial vaginosis.

Lactic acid is available in the market in the form of tablets, gels, suppositories, pessaries and marketed under various brand names like Fisiolat®, Gynofit®, Vagisan®, Lactofem, Premeno® duo, balance Activ®, VagiCare etc.

All the prior art references shows Lactic acid and low pH of the vaginal fluid create a selective antimicrobial barrier against harmful pathogens that can cause vaginal infections such as bacterial vaginosis, Lactic acid is used in different forms such as gel, lotions, tablets, suppositories, pessaries etc, antibacterial films that may regulate pH and control bacterial growth in vaginal environment, oligosaccharides promote the growth of beneficial Lactic acid bacteria in the colon, prebiotic compositions and formulations comprising Lactic acid as α-hydroxy acid, fructooligosaccharide as prebiotic agent in the form of liquid, solution, paste or gel. However, the inventors of the present invention have developed vaginal film composition comprising Lactic acid as active ingredient, optionally prebiotic and pharmaceutically acceptable excipients for prevention of urogenital tract infections. The inventors of present invention also provide process for the preparation of Lactic acid vaginal film comprising the steps of dissolving/melting, mixing, coating/coating & drying and cutting.

### OBJECTIVE OF INVENTION

The main objective of the present invention is to provide organic acid vaginal film composition.

Another objective of the present invention is to provide a composition of vaginal film comprising organic acid as active ingredient and pharmaceutically acceptable excipients.

Another objective of the present invention is to provide a composition of vaginal film comprising Lactic acid as organic acid, optionally prebiotics and pharmaceutically acceptable excipients.

Another objective of the present invention is to provide a composition of vaginal film comprising Lactic acid as active ingredient, optionally prebiotics and pharmaceutically acceptable excipients for prevention of urogenital tract infections.

Another objective of the present invention is to provide a composition of vaginal film comprising Lactic acid as active ingredient, optionally prebiotics and pharmaceutically acceptable excipients for prevention of urogenital tract infections, especially vaginal infections caused by elevated pH levels of vaginal cavity.

Another objective of the present invention is to provide a composition of vaginal film comprising Lactic acid as active ingredient, optionally prebiotics and pharmaceutically acceptable excipients for treating bacterial vaginosis.

Still another objective of the present invention is to provide a composition of vaginal film comprising Lactic acid as active ingredient, optionally fructooligosaccharides as prebiotic, film forming agent, plasticizers, neutralizing agent, lubricants as pharmaceutically acceptable excipients and preservatives, disintegrating agents, anti-foaming agent, antimicrobial agent, coloring agents, flavouring agents as optional excipients.

Still another objective of the present invention is to provide a composition of vaginal film comprising Lactic acid as active ingredient, fructooligosaccharides as prebiotics, film forming agents, plasticizers, neutralizing agent, lubricants, anti-foaming agent, antimicrobial agent, preservatives, disintegrating agents, coloring agents, flavouring agents as pharmaceutically acceptable excipients for prevention of urogenital tract infections.

Still another objective of the present invention is to provide process for the preparation of Lactic acid vaginal film comprising the steps of dissolving/melting, mixing, coating/coating & drying and cutting.

Still another objective of the present invention is to provide Lactic acid vaginal film which is having clinical advantage by improving patient compliance due to flexibility of the film, no leakage of drug from the film; such disadvantages are usually associated with suppositories. These films deliver precise dose and have no messiness while administration unlike gels, lotions, ointments and other liquid formulations.

In yet another objective of the present invention is to prevent urogenital tract infections by application of Lactic acid vaginal film.

### SUMMARY OF INVENTION

Accordingly, the present invention provides a composition of Lactic acid vaginal film useful in preventing urogenital tract infections.

Another embodiment of the present invention relates to a composition of organic acid vaginal film comprising Lactic acid as active ingredient, optionally prebiotics and pharmaceutically acceptable excipients for prevention of urogenital tract infections, especially vaginal infections caused by elevated pH levels of vaginal cavity.

Another embodiment of the present invention relates to a composition of organic acid vaginal film comprising Lactic acid as active ingredient, optionally prebiotics and pharmaceutically acceptable excipients for treating bacterial vaginosis.

In another embodiment, the present invention provides a composition of organic acid vaginal film comprising Lactic acid as active ingredient, optionally fructooligosaccharides as prebiotics, film forming agent, plasticizers, neutralizing agent, lubricants as pharmaceutically acceptable excipients and preservatives, disintegrating agents, anti-foaming agent, antimicrobial agent, coloring agents, flavouring agents as optional excipients.

In another embodiment, the present invention provides a composition of organic acid vaginal film comprising Lactic acid as active ingredient, fructooligosaccharides as prebiotics, film forming agents, plasticizers, neutralizing agent, lubricants, anti-foaming agent, antimicrobial agent, preservatives, disintegrating agents, coloring agents, flavouring agents as pharmaceutically acceptable excipients for prevention of urogenital tract infections.

In another embodiment, the present invention provides a composition of organic acid vaginal film comprising Lactic acid as active ingredient, optionally short chain fructooligosaccharides as prebiotics, eudragit EPO, copovidone, polyvinyl pyrrolidine, gelatine, poloxamer, hydroxypropyl cellulose, polyvinyl alcohol, hydroxypropyl methyl cellulose E 15, hydroxypropyl methylcellulose E50 as film forming agents, polyethylene glycol, propylene glycol, triethyl citrate, glycerin, vitamin E TPGS as plasticizers, sodium hydroxide as neutralizing agent, simethicone as anti-foaming agent, tea tree oil as antimicrobial agent, methylparaben, propylparaben as preservatives, stearic acid, silicon dioxide as lubricants, starch as disintegrating agents, titanium dioxide, FD&C, D&C, lakes, approved colours as coloring agents and acceptable flavours as flavouring agents.

In another embodiment, the present invention provides process for the preparation of Lactic acid vaginal film comprising the steps of dissolving/melting, mixing, coating/coating & drying and cutting.

In yet another embodiment, the present invention provides vaginal film comprising:

10% to 45% (w/w) of active ingredient,

3% to 10% (w/w) of prebiotics,

1% to 40% (w/w) of film forming agent,

1% to 40% (w/w) of plasticizer,

1% to 20% of lubricants, and

2% to 8% (w/w) of neutralizing agent.

In yet another embodiment, the present invention provides Lactic acid vaginal film comprising:

10% to 45% (w/w) of Lactic acid,

3% to 10% (w/w) of prebiotics optionally,

1% to 40% (w/w) of film forming agent,

1% to 40% (w/w) of plasticizer,

1% to 20% of lubricants,

2% to 8% (w/w) of neutralizing agent,

0.01% to 0.2% (w/w) of preservatives optionally,

3% to 7% (w/w) of disintegrating agent optionally,

0.05% to 0.5% (w/w) of anti-foaming agent optionally,

0.5% to 1% (w/w) of antimicrobial agent optionally,

0.0001% to 0.6% of coloring agents optionally, and

0.005% to 0.03% (w/w) of flavouring agent optionally.

In yet another embodiment, the present invention provides Lactic acid vaginal film comprising:

10% to 45% (w/w) of Lactic acid,

3% to 10% (w/w) of prebiotics,

1% to 40% (w/w) of film forming agent,

1% to 40% (w/w) of plasticizer,

1% to 20% of lubricants,

2% to 8% (w/w) of neutralizing agent,

0.01% to 0.2% (w/w) of preservatives,

3% to 7% (w/w) of disintegrating agent,

0.05% to 0.5% (w/w) of anti-foaming agent,

0.5% to 1% (w/w) of antimicrobial agent,

0.0001% to 0.6% of coloring agents, and

0.005% to 0.03% (w/w) of flavouring agent.

In still another embodiment, the present invention provides Lactic acid vaginal film which is having clinical advantage by improving patient compliance due to flexibility of the film, no leakage of drug from the film; such disadvantages are usually associated with suppositories. These films deliver precise dose and have no messiness while administration unlike gels, lotions, ointments and other liquid formulations.

In yet another embodiment, the present invention provides process for the preparation of Lactic acid vaginal film comprises dissolving active ingredient and neutralizing agent in solvent, adding optionally prebiotics, coloring agents, flavouring agent and preservatives, melting lubricants, plasticizers, film forming agents, adding disintegrating agents, adding molten mixture into obtained active ingredient mixture until uniform mixture is formed, coating, cooling, cutting, packing and sealing the films into sachets.

In yet another embodiment, the present invention provides a process for preparing vaginal film, the process comprising steps of:
a) dissolving active ingredient and neutralizing agent in solvent under continuous stirring and adding optionally prebiotics, coloring agents, flavouring agent, anti-foaming agent, antimicrobial agent and preservatives,
b) melting one or more lubricants, plasticizers and film forming agents under heating and continuous stirring and adding disintegrating agents,
c) adding step (a) to step (b) under continuous heating, stirring and mixing it until uniform mixture is formed,
d) coating the above solution on suitable liner like polyethylene terephthalate by using hot melt coater at 80 to 140° C.,
e) cooling down the coat to room temperature, and
f) cutting into desired size to get vaginal film, packing and sealing the film into sachets.

In yet another embodiment, the present invention provides a process for preparing vaginal film, the process comprising steps of:
a) dissolving Lactic acid and sodium hydroxide in demineralized water under continuous stirring and adding optionally fructooligosaccharides, coloring agents, flavouring agent, simethicone, tea tree oil, methylparaben and propylparaben,
b) melting stearic acid and/or silicon dioxide, polyethylene glycol or triethyl citrate or vitamin E TPGS or glycerine and eudragit or gelatine or copovidone or polyvinyl pyrrolidine or hydroxy propyl cellulose or poloxamer under heating and continuous stirring and adding starch,
c) adding step (a) to step (b) under continuous heating, stirring and mixing it until uniform mixture is formed,
d) coating the above solution on suitable liner like polyethylene terephthalate by using hot melt coater at 80 to 140° C.,
e) cooling down the coat to room temperature, and
f) cutting into desired size to get vaginal film, packing and sealing the film into sachets.

In yet another embodiment, the present invention provides process for the preparation of Lactic acid vaginal film comprises dissolving active ingredient and neutralizing agent in solvent, adding lubricants, disintegrating agents, plasticizers, coloring agents, flavouring agent, preservatives under stirring, adding film forming agent under stirring, coating, drying, cutting, packing and sealing the films into sachets.

In yet another embodiment, the present invention provides a process for preparing vaginal film, the process comprising steps of:
a) dissolving active ingredient and neutralizing agent in solvent under continuous stirring,
b) adding one or more lubricants, disintegrating agents, plasticizers, coloring agents, flavouring agent and preservatives to step (a) under continuous stirring,
c) adding film forming agent to step (b) under continuous stirring and mixing it until uniform mixture is formed,
d) coating the above solution on suitable liner like polyethylene terephthalate by using film applicator,
e) drying the coated liquid polymeric film in hot air oven, and
f) cutting into desired size to get vaginal film, packing and sealing the film into sachets.

In yet another embodiment, the present invention provides a process for preparing vaginal film, the process comprising steps of:
a) dissolving Lactic acid and sodium hydroxide in demineralized water under continuous stirring,
b) adding silicon dioxide, starch, propylene glycol or glycerin, colorant, flavor, methylparaben and propylparaben to step (a) under continuous stirring,
c) adding polyvinyl alcohol or hydroxypropyl methylcellulose to step (b) under continuous stirring and mixing it until uniform mixture is formed,
d) coating the above solution on suitable liner like polyethylene terephthalate by using film applicator,
e) drying the coated liquid polymeric film in hot air oven at 50 to 80° C. for 45 to 180 minutes, and
f) cutting into desired size to get vaginal film, packing and sealing the film into sachets.

In yet another embodiment, the present invention provides process for the preparation of Lactic acid vaginal film comprises dissolving active ingredient and neutralizing agent in solvent, adding one or more prebiotics, lubricants, disintegrating agents, plasticizers, coloring agents, flavouring agent, preservatives under stirring, dispersing film forming agent in a solvent in separate vessel under stirring, adding dispersed film forming agent to the obtained mixture, coating, drying, cutting, packing and sealing the films into sachets.

In yet another embodiment, the present invention provides a process for preparing vaginal film, the process comprising steps of:
a) dissolving active ingredient and neutralizing agent in solvent under continuous stirring,
b) adding one or more prebiotics, lubricants, disintegrating agents, plasticizers, coloring agents, flavouring agent and preservatives to step (a) under continuous stirring,
c) dispersing film forming agent in solvent in a separate vessel under continuous stirring and cooling it,
d) adding step (b) into step (c) with continuous stirring until uniform mixture is formed,
e) coating the above solution on suitable liner like polyethylene terephthalate by using film applicator,
f) drying the coated liquid polymeric film in hot air oven, and
g) cutting into desired size to get vaginal film, packing and sealing the film into sachets.

In yet another embodiment, the present invention provides a process for preparing vaginal film, the process comprising steps of:
a) dissolving Lactic acid and sodium hydroxide in demineralized water under continuous stirring,
b) adding fructooligosaccharides, silicon dioxide, starch, propylene glycol or glycerin, colorant, flavor, methylparaben and propylparaben to step (a) under continuous stirring,
c) dispersing polyvinyl alcohol or hydroxypropyl methylcellulose into purified water at 70° C. in a separate vessel under continuous stirring and cooling to 50° C.,
d) adding step (b) into step (c) with continuous stirring until uniform mixture is formed,
e) coating the above solution on suitable liner like polyethylene terephthalate by using film applicator,
f) drying the coated liquid polymeric film at average temperature of 70 to 80° C. for 30 to 90 minutes, and
g) cutting into desired size to get vaginal film, packing and sealing the film into sachets.

In yet another embodiment, the present invention provides a process for preparing vaginal film, the process comprising steps of:
a) dissolving active ingredient and neutralizing agent in solvent under continuous stirring,
b) adding one or more prebiotics, lubricants, disintegrating agents, antifoaming agent, antimicrobial agent, plasticizers, coloring agents, flavouring agent and preservatives to step (a) under continuous stirring,
c) dispersing film forming agent in solvent in a separate vessel under continuous stirring and cooling it,
d) adding step (b) into step (c) with continuous stirring until uniform mixture is formed,
e) coating the above solution on suitable liner like polyethylene terephthalate by using film applicator,
f) drying the coated liquid polymeric film in hot air oven, and
g) cutting into desired size to get vaginal film, packing and sealing the film into sachets.

In yet another embodiment, the present invention provides a process for preparing vaginal film, the process comprising steps of:
a) dissolving Lactic acid and sodium hydroxide in demineralized water under continuous stirring,
b) adding fructooligosaccharides, silicon dioxide, starch, simethicone, tea tree oil, propylene glycol or glycerin, colorant, flavor, methylparaben and propylparaben to step (a) under continuous stirring,
c) dispersing polyvinyl alcohol or hydroxypropyl methylcellulose into purified water at 70° C. in a separate vessel under continuous stirring and cooling to 50° C.,
d) adding step (b) into step (c) with continuous stirring until uniform mixture is formed,
e) coating the above solution on suitable liner like polyethylene terephthalate by using film applicator,
f) drying the coated liquid polymeric film at average temperature of 70 to 80° C. for 30 to 90 minutes, and
g) cutting into desired size to get vaginal film, packing and sealing the film into sachets.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

Vagina has a powerful yet simple and ingenious protection system to prevent from the disorderly and problem-causing proliferation of germs. The vaginal protection system is based on the maintenance of a 3.5 to 4.5 acidic pH where the common and potentially pathogenic saprophytes do not find the favourable conditions to proliferate, whereas acidophilus bacilli are in their ideal environment. In these circumstances, acidophilus bacilli highly metabolize and produce large amounts of Lactic acid by glucose anaerobic fermentation.

Lactic acid keeps its acidic pH, thus creating a virtuous circle that prevents the uncontrolled growth of other bacteria present in the vagina. The origin of vaginitis and vaginosis lies in the breaking of the “acidic pH-acidophilic bacteria high metabolism-Lactic acid production-acidic pH” virtuous circle. The reasons why this circle is broken are many: use of broad-spectrum antibiotics, inadequate douches, long periods, etc. and the consequences are the following: vagina alkalization, slowing down of acidophilus bacilli metabolism, and decrease in Lactic acid production. A vicious circle is created, where the usually harmless vaginal saprophytic bacteria find an appropriate medium to proliferate and where vaginitis or vaginosis typical profiles are triggered.

Exogenous addition of Lactic acid leads to the acidification of the vagina, making it easier for Lactic acid bacilli to regain their metabolic capacity. When this happens, a virtuous circle created by Lactic acid bacilli begins. Indeed, these increase the production of Lactic acid, which acidifies the vagina. Then, the existence Lactic acid creates an adverse environment for the growth of pathogens, which number gets eventually drastically reduced. When this occurs, vaginal infection/odor/discomfort disappears.

The vaginal films pharmaceutical form consists of a thin and small sheet of polymeric hydrophilic substances that disperse or dissolve in contact with vaginal fluids to release the active substance. Vaginal films are pharmaceutical forms that are convenient, discrete, they do not require the use of an applicator to be administered and, as they disperse in vaginal fluids, they have low risk of increasing fluids volume and leak out, therefore improving comfort of use and efficacy after administration. Other advantages of vaginal films include their portability and low cost per unit. Since they are solid pharmaceutical forms, films can vehicle drugs that are susceptible to hydrolytic degradation, guaranteeing their stability. They can be formulated for immediate or sustained drug release.

The term “vaginal film” of the present invention can be interchangeably used with “vaginal device”.

The present invention provides vaginal film comprising organic acid as active ingredient, optionally prebiotics and pharmaceutically acceptable excipients.

The present invention provides vaginal film comprising Lactic acid as active ingredient, optionally prebiotics and pharmaceutically acceptable excipients.

The term “active ingredients” of the present invention is used to prevent urogenital tract infections, especially vaginal infections caused by elevated pH levels of vaginal cavity and also for treating bacterial vaginosis. Preferably used active ingredient is organic acid. More preferably used active ingredient is Lactic acid. Most preferably used active ingredient is Lactic acid (90%).

The concentration of active ingredient used in the vaginal film of present invention is from 10% to 45% (w/w). Preferably used concentration of active ingredient is from 23.28% to 39.29% (w/w).

The term “prebiotics” of the present invention includes fructo-oligosaccharides preferably a short-chain fructo-oligosaccharide derived from beets or sugar cane, will provides supplement for the growth of vaginal flora including Lactic acid bacilli. Use of prebiotics in vaginal film formulation is to promote the growth of native vaginal bacteria especially Lactic acid producing bacteria strains.

Prebiotics used in the composition of the present invention includes but not limited to inulin, fructooligosaccharides (FOS), galactooligosaccharides (GOS), lactulose, polydextrose, isomaltooligosaccharides (IMO), xylooligosaccahrides (XOS), lactitol, chicory root inulin-derived, wheat bran-derived arabinoxylooligosaccharides (AXOS), xylooligosaccharides (XOS). Prebiotics can also be found in some vegetables, such as leeks, onions, chicory, tomatoes, asparagus, artichokes, bananas and alfalfa. Preferably used prebiotics are fructooligosaccharides. Most preferably used prebiotics are short-chain fructo-oligosaccharides.

The functional properties of fructooligosaccharides (FOS), such as the reduction of cholesterol levels and blood glucose levels, lowering of blood pressure, better absorption of calcium and magnesium and to inhibit production of the reductase enzymes that can contribute to cancer. FOS are not digested by the human gastrointestinal tract, and when they reach the colon, they beneficially stimulate the growth and strengthening of specific bacteria in the intestine.

The concentration of prebiotics used in the vaginal film of the present invention is from 3% to 10% (w/w). Preferably used concentration of prebiotics is from 3.11% to 7.14% (w/w).

The term “film forming agents” as used herein includes but not limited to hydroxyethyl cellulose, carrageen, eudragit EPO, hydroxypropyl cellulose, hydroxypropyl methylcellulose, gums, starch, polymerized rosin, pullulan, sodium alginate, pectin, gelatine, chitosan, maltodextrins, polyvinyl alcohol, sodium carboxy methyl cellulose, copovidone, polyvinyl pyrrolidone, poloxamer, ammonium alginate, ethyl cellulose, ethyl lactate, hydroxy propyl methyl cellulose acetate succinate, poly ethylene oxide, higher molecular weight poly ethylene glycols, poly methacrylates, poly(methylvinyl ether/maleic anhydride, polyvinyl acetate phthalate and shellac. Most preferably used film forming agent are eudragit EPO, copovidone, polyvinyl pyrrolidine, gelatine, poloxamer, hydroxypropyl cellulose, polyvinyl alcohol, hydroxypropyl methyl cellulose E15 and hydroxypropyl methylcellulose E50.

The concentration of film forming agent used individually in the vaginal film of the present invention is from 1% to 40% (w/w). Preferably used concentration of film forming agent individually is from 1.56% to 37.84%. The concentration of film forming agent used in combination in the vaginal film of the present invention is from 15% to 50% (w/w). Preferably used concentration of film forming agent in combination is from 28.40% to 46.77%.

Plasticizers are low molecular weight compounds that can be added to the formulation to increase the plasticity, soften the polymer carrier, and enhance the flexibility of the final product. The addition of plasticizers to the formulation can improve the manufacturing conditions or the physiochemical properties of the film.

Plasticizers used in the composition of the present invention include but not limited to triacetin, low molecular weight polyethylene glycols, triethyl citrate, glycerine, propylene glycol, acetyltributyl citrate, acetyltriethyl citrate, benzyl benzoate, cellulose acetate phthalate compatible, chlorbutanol, dextrin, dibutyl phthalate, dibutyl sebacate, diethyl phthalate, dimethyl phthalate, glycerin monostearate, mannitol, mineral oil and lanolin alcohols, palmitic acid, petrolatum and lanolin alcohols, pyrrolidone, sorbitol, tributyl citrate, triethanolamine and vitamin E TPGS. Preferably used plasticizers are polyethylene glycol, propylene glycol, triethyl citrate, vitamin E TPGS and glycerin.

Propylene glycol and glycerin were used as plasticizer and hygroscopic material to prevent moisture loss.

The concentration of plasticizer used in the vaginal film of present invention is from 1% to 40% (w/w). Preferably used concentration of plasticizer individually is from 1.08% to 37.04% (w/w). The concentration of plasticizers used in combination in the vaginal film of the present invention is from 2% to 20% (w/w). Preferably used concentration of plasticizers is from 3.12% to 18.28% (w/w).

Neutralizing agent used in the composition of the present invention include but not limited to sodium carbonate, sodium bicarbonate, potassium carbonate, potassium bicarbonate, sodium phosphate dibasic, sodium phosphate tribasic, potassium phosphate dibasic, potassium phosphate tribasic, calcium carbonate, magnesium carbonate, sodium hydroxide, magnesium hydroxide, potassium hydroxide, aluminum hydroxide, and combinations thereof. Preferably used neutralizing agent is sodium hydroxide.

The concentration of neutralizing agent used in the vaginal film of present invention is from 2% to 8% (w/w). Most preferably used concentration of neutralizing agent is from 3.23% to 5.36% (w/w).

Preservatives are used to inhibit the growth of microorganisms over an extended period of time. Preservatives used in the composition of the present invention include but not limited to alkanols, disodium EDTA (ethylenediamine tetraacetate), EDTA salts, EDTA fatty acid conjugates, isothiazolinone, benzoic esters (parabens) (e.g., methylparaben, propylparaben, butylparaben, ethylparaben, isopropylparaben, isobutylparaben, benzylparaben, sodium methylparaben, and sodium propylparaben), benzoic acid, propylene glycols, sorbates and urea derivatives (e.g., diazolindinyl urea). Preferably used preservatives are methylparaben and propylparaben.

The concentration of preservative used in the vaginal film of present invention is from 0.01% to 0.2% (w/w). Preferably used concentration of preservative individually is from 0.02% to 0.13% (w/w). Preferably used concentration of preservative in combination is from 0.14 to 0.16% (w/w) of the total weight of the composition.

Lubricants used in the composition of the present invention include but not limited to stearic acid, calcium stearate, glycerin monostearate, glyceryl behenate, glyceryl palmitostearate, hydrogenated castor oil, light mineral oil, magnesium lauryl sulfate, magnesium stearate, medium-chain triglycerides, mineral oil, myristic acid, palmitic acid, sodium benzoate, sodium lauryl sulfate, sodium stearyl fumarate, talc, zinc stearate and silicon dioxide.

The concentration of lubricant used in the vaginal film of present invention is from 1% to 20% (w/w). Most preferably used concentration of lubricant is from 2.82% to 18.75% (w/w).

Lactic acid vaginal film composition and process of the present invention further includes anti-foaming agents, antimicrobial agents, disintegrating agents, coloring agents and flavouring agents.

Anti-foaming agents used in the composition of the present invention include but not limited to simethicone, alcohols like cetostearyl alcohol, insoluble oils (castor oil), oleic acid, stearates, other silicones derivatives, ether, glycols, 2-octanol, paraffinic waxes, amide waxes, sulfonated oils, organic phosphates, silicone oils and mineral oils. Preferably used anti-foaming agent is Simethicone.

The concentration of anti-foaming agent used in the vaginal film of present invention is from 0.05% to 0.5% (w/w). Preferably used concentration of anti-foaming agent is from 0.08% to 0.31% (w/w) of the total weight of the composition.

Antimicrobial agent used in the composition of the present invention includes but not limited to oregano oil, basil oil, rosemarin oil, eucalyptus oil, tea tree oil, or thyme oil. Preferably used antimicrobial agent is tea tree oil.

The concentration of antimicrobial agent used in the vaginal film of present invention is from 0.5% to 1% (w/w). Preferably used concentration of anti-foaming agent is 0.71% (w/w) of the total weight of the composition.

Preferably used disintegrating agent is starch, lactose, monohydrate, corn starch, alginic acid, calcium alginate, cellulose, powdered, croscarmellose sodium, crospovidone, docusate sodium, glycine, magnesium aluminum silicate, methylcellulose, microcrystalline cellulose, polacrilin potassium, sodium alginate and sodium starch glycolate.

The concentration of disintegrating agents used in the vaginal film of present invention is from 3% to 7% (w/w). Most preferably used concentration of disintegrating agents is from 3.89% to 5.63% (w/w).

Preferably used coloring agents are titanium dioxide, FD&C, D&C, lakes and any approved colour/colorants. The concentration of coloring agents used in the vaginal film of present invention is from 0.0001% to 0.6% (w/w). Most preferably used concentration of coloring agents is from 0.0003% to 0.53% (w/w).

Flavouring agents used in the composition of the present invention include any acceptable flavour used in vaginal film.

The concentration of flavouring agents used in the vaginal film of present invention is from 0.005% to 0.03% (w/w). Most preferably used concentration of flavouring agents is from 0.007% to 0.01% (w/w).

The present invention provides composition of vaginal film comprising Lactic acid as organic acid, optionally fructooligosaccharides as prebiotic film forming agent, plasticizers, neutralizing agent, lubricants as pharmaceutically acceptable excipients and preservatives, disintegrating agents, coloring agents, flavouring agents as optional excipients.

The present invention provides composition of organic acid vaginal film comprising Lactic acid as active ingredient, optionally short chain fructooligosaccharides as prebiotics, eudragit EPO, copovidone, polyvinyl pyrrolidine, gelatine, poloxamer, hydroxypropyl cellulose, polyvinyl alcohol, hydroxypropyl methyl cellulose E 15, hydroxypropyl methylcellulose E50 as film forming agents, polyethylene glycol, propylene glycol, triethyl citrate, glycerin, vitamin E TPGS as plasticizers, sodium hydroxide as neutralizing agent, methylparaben, propylparaben as preservatives, stearic acid, silicon dioxide as lubricants, starch as disintegrating agents, titanium dioxide, FD&C, D&C, lakes, approved colours as coloring agents.

The present invention is to provide process for the preparation of Lactic acid vaginal film comprising the steps of dissolving/melting, mixing, coating/coating & drying and cutting.

The present invention is to provide process for the preparation of Lactic acid vaginal film dissolving active ingredient and neutralizing agent in solvent, adding lubricants, disintegrating agents, plasticizers, coloring agents, flavouring agent, preservatives under stirring, adding film forming agent under stirring, coating, drying, cutting, packing and sealing the films into sachets.

The present invention is to provide process for the preparation of Lactic acid vaginal film dissolving active ingredient and neutralizing agent in solvent, adding lubricants, disintegrating agents, plasticizers, coloring agents, flavouring agent, preservatives under stirring, dispersing film forming agent in a solvent in separate vessel under stirring, adding dispersed film forming agent to the obtained mixture, coating, drying, cutting, packing and sealing the films into sachets.

The present invention is to provide Lactic acid vaginal film which is having clinical advantage by improving patient compliance due to flexibility of the film, no leakage of drug from the film; such disadvantages are usually associated with suppositories. These films deliver precise dose and have no messiness while administration unlike gels, lotions, ointments and other liquid formulations.

The following examples describes the nature of the invention and are given only for the purpose of illustrating the present invention in more detail and are not limitative and relate to solutions which have been particularly effective on a bench scale and prepared by the process of the present invention.

### Manufacturing Process 1:

Required quantities of Lactic acid and sodium hydroxide pellets were added to demineralized water and stirred continuously until they dissolve completely. Required quantities of prebiotics, antifoaming agent, antimicrobial agent, coloring agent, flavouring agent and preservatives were added optionally and stirred continuously. Required quantities of lubricants, plasticizers, and film forming agents were heated separately up to molten state and disintegrating agents were added. Prepared Lactic acid solution was added to the obtained molten mixture, mixed and stirred continuously under heating until uniform mixture was formed. Obtained solution was coated on suitable liner like polyethylene terephthalate by using hot melt coater at 80 to 140° C. Coated liquid was cooled down to room temperature. The resultant was cut into desired size of films. Obtained Lactic acid vaginal films were packed and sealed in suitable sachets.

### Manufacturing Process 2:

Required quantities of Lactic acid and sodium hydroxide pellets were added to demineralized water and stirred continuously until they dissolve completely. Required quantities of lubricants, disintegrating agents, plasticizers, coloring agent, flavouring agent and preservatives were added and stirred continuously and film forming agent was added to the obtained mixture and stirred continuously until uniform mixture was formed. Obtained solution was coated on suitable liner like polyethylene terephthalate by using film applicator. Coated liquid polymeric film was dried in hot air oven at 50° C. to 80° C. for 45 to 180 minutes. The resultant was cut into desired size of films. Obtained Lactic acid vaginal films were packed and sealed in suitable sachets.

### Manufacturing Process 3:

Required quantities of Lactic acid and sodium hydroxide pellets were added to demineralized water and stirred continuously until they dissolve completely. Required quantities of one or more prebiotics, lubricants, disintegrating agents, plasticizers, coloring agent, flavouring agent and preservatives were added, stirred continuously and film forming agent was dispersed in a solvent at 70° C. in separate vessel, stirred continuously, cooled to 50° C. and added to the obtained mixture and stirred continuously until uniform mixture was formed. Obtained solution was coated on suitable liner like polyethylene terephthalate by using film applicator. Coated liquid polymeric film was dried in hot air oven at 70° C. to 80° C. for 30 to 90 minutes. The resultant was cut into desired size of films. Obtained Lactic acid vaginal films were packed and sealed in suitable sachets.

### Manufacturing Process 4:

Required quantities of Lactic acid and sodium hydroxide pellets were added to demineralized water and stirred continuously until they dissolve completely. Required quantities of one or more prebiotics, lubricants, disintegrating agents, plasticizers, anti-foaming agent, antimicrobial agents, coloring agent, flavouring agent and preservatives were added, stirred continuously and film forming agent was dispersed in a solvent at 70° C. in separate vessel, stirred continuously, cooled to 50° C. and added to the obtained mixture and stirred continuously until uniform mixture was formed. Obtained solution was coated on suitable liner like polyethylene terephthalate by using film applicator. Coated liquid polymeric film was dried in hot air oven at 70° C. to 80° C. for 30 to 90 minutes. The resultant was cut into desired size of films Obtained Lactic acid vaginal films were packed and sealed in suitable sachets.

### EXAMPLES

### Example 1

| | | | Amount per |
|---|---|---|---|
| S. No | Ingredients | Quantity (% w/w) | strip in mg |
| 1. | Lactic acid | 29.73 | 220.0 |
| 2. | Stearic acid | 13.51 | 100.0 |
| 3. | Polyethylene Glycol | 9.46 | 70.0 |
| 4. | Eudragit EPO | 37.84 | 280.0 |
| 5. | Starch 1500 | 5.41 | 40.0 |
| 6. | Approved colour | 0.003 | 0.02 |
| 7. | Purified water | 4.05 | 30.0 |
| 8. | Total | | 710.02 |

### Example 2

| | | | Amount per |
|---|---|---|---|
| S. No | Ingredients | Quantity (% w/w) | strip in mg |
| 1. | Lactic acid | 23.28 | 220.0 |
| 2. | Fructooligosaccharide | 4.23 | 40.0 |
| 3. | Stearic acid | 10.58 | 100.0 |
| 4. | Polyethylene Glycol | 7.41 | 70.0 |
| 5. | Eudragit EPO | 27.51 | 260.0 |
| 6. | Starch 1500 | 4.23 | 40.0 |
| 7. | Sodium hydroxide | 4.23 | 40.00 |
| 8. | Triethyl citrate | 4.23 | 40.0 |
| 9. | Silicon dioxide | 10.58 | 100.0 |
| 10. | Titanium dioxide | 0.53 | 5.0 |
| 11. | Approved colour | 0.002 | 0.02 |
| 12. | Purified water | 3.17 | 30.0 |
| | Total | | 915.02 |

### Example 3

| | | | Amount per |
|---|---|---|---|
| S. No | Ingredients | Quantity (% w/w) | strip in mg |
| 1. | Lactic acid | 27.50 | 220.0 |
| 2. | Stearic acid | 18.75 | 150.0 |
| 3. | Polyethylene Glycol | 18.75 | 150.0 |
| 4. | Eudragit EPO | 25.00 | 200.0 |
| 5. | Starch 1500 | 5.00 | 40.0 |
| 6. | Purified water | 5.00 | 40.0 |
| | Total | | 760.0 |

### Example 4

| | | | Amount per |
|---|---|---|---|
| S. No | Ingredients | Quantity (% w/w) | strip in mg |
| 1. | Lactic acid | 27.16 | 220.0 |
| 2. | Polyethylene Glycol | 37.04 | 300.0 |
| 3. | Eudragit EPO | 24.69 | 200.0 |
| 4. | Starch 1500 | 4.94 | 40.0 |
| 5. | Purified water | 6.17 | 50.0 |
| | Total | | 760.0 |

### Example 5

| | | | Amount per |
|---|---|---|---|
| S. No | Ingredients | Quantity (% w/w) | strip in mg |
| 1. | Lactic acid | 25.00 | 220.0 |
| 2. | Fructooligosaccharide | 4.55 | 40.0 |
| 3. | Stearic acid | 11.36 | 100.0 |
| 4. | Polyethylene Glycol | 7.95 | 70.0 |
| 5. | Eudragit EPO | 22.73 | 200.0 |
| 6. | Starch 1500 | 4.55 | 40.0 |
| 7. | Sodium hydroxide | 4.55 | 40.0 |
| 8. | Copovidone | 6.82 | 60.0 |
| 9. | Glycerine | 3.41 | 30.0 |
| 10. | Approved colour | 0.002 | 0.02 |
| 11. | Purified water | 9.09 | 79.99 |
| | Total | | 800.02 |

### Example 6

| | | | Amount per |
|---|---|---|---|
| S. No | Ingredients | Quantity (% w/w) | strip in mg |
| 1. | Lactic acid | 24.44 | 220.0 |
| 2. | Fructooligosaccharide | 4.44 | 40.0 |
| 3. | Stearic acid | 11.11 | 100.0 |
| 4. | Polyethylene Glycol | 7.78 | 70.0 |
| 5. | Eudragit EPO | 22.22 | 200.0 |
| 6. | Starch 1500 | 4.44 | 40.0 |
| 7. | Sodium hydroxide | 4.44 | 39.97 |
| 8. | Polyvinyl pyrrolidine | 6.67 | 60.04 |
| 9. | Glycerine | 3.33 | 30.0 |
| 10. | Approved colour | 0.002 | 0.02 |
| 11. | Purified water | 11.11 | 100.01 |
| | Total | | 800.03 |

### Example 7

| | | | Amount per |
|---|---|---|---|
| S. No | Ingredients | Quantity (% w/w) | strip in mg |
| 1. | Lactic acid | 26.51 | 220.0 |
| 2. | Fructooligosaccharide | 4.82 | 40.00 |
| 3. | Stearic acid | 6.02 | 50.0 |
| 4. | Polyethylene Glycol | 8.43 | 70.0 |
| 5. | Gelatine | 15.66 | 130.0 |
| 6. | Poloxamer | 15.66 | 130.0 |
| 7. | Starch 1500 | 4.82 | 40.00 |
| 8. | Sodium hydroxide | 3.61 | 30.0 |
| 9. | Approved colour | 0.002 | 0.02 |
| 10. | Purified water | 14.46 | 120.00 |
| | Total | | 710.02 |

### Example 8

| S. | | Quantity | Amount per |
|---|---|---|---|
| No | Ingredients | (% w/w) | strip in mg |
| 1. | Lactic acid | 26.83 | 220.0 |
| 2. | Fructooligosaccharide | 4.88 | 40.01 |
| 3. | Stearic acid | 6.10 | 50.0 |
| 4. | Polyethylene Glycol | 8.54 | 70.0 |
| 5. | Eudragit EPO | 26.83 | 220.0 |
| 6. | Gelatine | 7.32 | 60.0 |
| 7. | Starch 1500 | 4.88 | 40.01 |
| 8. | Sodium hydroxide | 3.66 | 30.0 |
| 9. | Approved colour | 0.002 | 0.02 |
| 10. | Purified water | 10.98 | 90.03 |
| | Total | | 730.04 |

### Example 9

| S. | | Quantity | Amount per |
|---|---|---|---|
| No | Ingredients | (% w/w) | strip in mg |
| 1. | Lactic acid | 23.91 | 220.0 |
| 2. | Fructooligosaccharide | 4.35 | 40.03 |
| 3. | Stearic acid | 5.43 | 50.0 |
| 4. | Polyethylene Glycol | 7.61 | 70.0 |
| 5. | Gelatine | 14.13 | 130.0 |
| 6. | Poloxamer | 14.13 | 130.0 |
| 7. | Starch 1500 | 4.35 | 40.03 |
| 8. | Sodium hydroxide | 3.26 | 30.0 |
| 9. | Copovidone | 6.52 | 59.99 |
| 10. | Glycerine | 1.09 | 10.03 |
| 11. | Approved colour | 0.002 | 0.02 |
| 12. | Purified water | 15.22 | 140.04 |
| | Total | | 780.10 |

### Example 10

| S. | | Quantity | Amount per |
|---|---|---|---|
| No | Ingredients | (% w/w) | strip in mg |
| 1. | Lactic acid | 23.66 | 220.0 |
| 2. | Fructooligosaccharide | 4.30 | 39.99 |
| 3. | Stearic acid | 5.38 | 50.0 |
| 4. | Polyethylene Glycol | 7.53 | 70.0 |
| 5. | Gelatine | 13.98 | 130.0 |
| 6. | Poloxamer | 13.98 | 130.0 |
| 7. | Starch 1500 | 4.30 | 39.98 |
| 8. | Sodium hydroxide | 3.23 | 30.0 |
| 9. | Polyvinyl pyrrolidine | 6.45 | 59.97 |
| 10. | Glycerine | 1.08 | 10.04 |
| 11. | Approved colour | 0.002 | 0.02 |
| 12. | Purified water | 13.98 | 129.99 |
| | Total | | 780.00 |

### Example 11

| S. | | Quantity | Amount per |
|---|---|---|---|
| No | Ingredients | (% w/w) | strip in mg |
| 1. | Lactic acid | 24.72 | 220.0 |
| 2. | Fructooligosaccharide | 4.49 | 39.97 |
| 3. | Stearic acid | 5.62 | 50.0 |
| 4. | Polyethylene Glycol | 7.87 | 70.0 |
| 5. | Gelatine | 14.61 | 130.0 |
| 6. | Poloxamer | 14.61 | 130.0 |
| 7. | Starch 1500 | 4.49 | 39.96 |
| 8. | Sodium hydroxide | 3.37 | 30.0 |
| 9. | Hydroxy propyl cellulose | 6.74 | 59.99 |
| 10. | Glycerine | 1.12 | 9.97 |
| 11. | Approved colour | 0.002 | 0.02 |
| 12. | Purified water | 12.36 | 110.00 |
| | Total | | 780.00 |

### Example 12

| S. | | Quantity | Amount per |
|---|---|---|---|
| No | Ingredients | (% w/w) | strip in mg |
| 1. | Lactic acid | 24.18 | 220.0 |
| 2. | Fructooligosaccharide | 4.40 | 40.03 |
| 3. | Stearic acid | 5.49 | 50.0 |
| 4. | Polyethylene Glycol | 7.69 | 70.0 |
| 5. | Eudragit EPO | 10.99 | 100.0 |
| 6. | Gelatine | 6.59 | 60.0 |
| 7. | Poloxamer | 14.29 | 130.02 |
| 8. | Starch 1500 | 4.40 | 40.0 |
| 9. | Sodium hydroxide | 3.30 | 30.02 |
| 10. | Copovidone | 3.30 | 30.02 |
| 11. | Polyvinyl pyrrolidine | 3.30 | 30.02 |
| 12. | Glycerine | 1.10 | 10.01 |
| 13. | Approved colour | 0.002 | 0.02 |
| 14. | Purified water | 10.99 | 99.99 |
| | Total | | 810.14 |

### Example 13

| S. | | Quantity | Amount per |
|---|---|---|---|
| No | Ingredients | (% w/w) | strip in mg |
| 1. | Lactic acid | 23.66 | 220.0 |
| 2. | Fructooligosaccharides | 4.30 | 40.0 |
| 3. | Stearic acid | 10.75 | 100.0 |
| 4. | Polyethylene Glycol | 7.53 | 70.0 |
| 5. | Eudragit EPO | 21.51 | 200.0 |
| 6. | Starch 1500 | 4.30 | 40.0 |
| 7. | Sodium hydroxide | 3.23 | 30.0 |
| 8. | Gelatine | 8.60 | 80.00 |
| 9. | Vitamin E TPGS | 10.75 | 99.96 |
| 10. | Approved colour | 0.002 | 0.02 |
| 11. | Purified water | 5.38 | 50.03 |
| | Total | | 880.00 |

### Example 14

| S. | | Quantity | Amount per |
|---|---|---|---|
| No | Ingredients | (% w/w) | strip in mg |
| 1. | Lactic acid | 34.92 | 220.0 |
| 2. | Fructooligosaccharides | 6.35 | 40.0 |
| 3. | Sodium hydroxide | 4.76 | 30.0 |
| 4. | Polyvinyl alcohol | 34.77 | 219.1 |
| 5. | Silicon dioxide | 3.17 | 20.0 |
| 6. | Maize starch | 4.76 | 29.99 |
| 7. | Methylparaben | 0.12 | 0.8 |
| 8. | Propylparaben | 0.02 | 0.13 |
| 9. | Glycerine | 3.17 | 19.97 |
| 10. | Approved colour | 0.003 | 0.02 |
| 11. | Purified water | 7.94 | 50.02 |
| | Total | | 580.01 |

### Example 15

| S. | | Quantity | Amount per |
|---|---|---|---|
| No | Ingredients | (% w/w) | strip in mg |
| 1. | Lactic acid | 37.93 | 220.0 |
| 2. | Fructooligosaccharides | 6.90 | 40.0 |
| 3. | Sodium hydroxide | 5.17 | 30.0 |
| 4. | Polyvinyl alcohol | 23.98 | 139.1 |
| 5. | Hydroxypropyl methyl | 6.90 | 40.0 |
| | cellulose E 15 | | |
| 6. | Silicon dioxide | 3.45 | 20.01 |
| 7. | Maize starch | 5.17 | 30.0 |
| 8. | Methylparaben | 0.13 | 0.75 |
| 9. | Propylparaben | 0.03 | 0.17 |
| 10. | Glycerine | 3.45 | 20.01 |
| 11. | Approved colour | 0.003 | 0.02 |
| 12. | Purified water | 6.90 | 40.02 |
| | Total | | 540.06 |

### Example 16

| S. | | Quantity | Amount per |
|---|---|---|---|
| No | Ingredients | (% w/w) | strip in mg |
| 1. | Lactic acid | 39.29 | 220.0 |
| 2. | Fructooligosaccharides | 7.14 | 40.0 |
| 3. | Sodium hydroxide | 5.36 | 30.0 |
| 4. | Polyvinyl alcohol | 21.26 | 119.0 |
| 5. | Hydroxypropyl methyl | 7.14 | 40.0 |
| | cellulose E 50 | | |
| 6. | Silicon dioxide | 3.57 | 20.0 |
| 7. | Maize starch | 5.36 | 30.0 |
| 8. | Methylparaben | 0.13 | 0.75 |
| 9. | Propylparaben | 0.03 | 0.15 |
| 10. | Glycerine | 3.57 | 20.00 |
| 11. | Approved colour | 0.002 | 0.02 |
| 12. | Purified water | 7.14 | 39.98 |
| | Total | | 519.92 |

### Example 17

| S. | | Quantity | Amount per |
|---|---|---|---|
| No | Ingredients | (% w/w) | strip in mg |
| 1. | Lactic acid (90%) | 36.37 | 110 |
| 2. | Fructooligosaccharides | 6.67 | 20 |
| 3. | Hydroxypropyl | 34.84 | 104.509 |
| | methylcellulose E50 | | |
| 4. | Propylene glycol | 3.33 | 10 |
| 5. | Glycerin | 3.33 | 10 |
| 6. | Maize starch | 5 | 15 |
| 7. | Silicon dioxide | 5 | 15 |
| 8. | Sodium hydroxide | 5 | 15 |
| 9. | Methylparaben | 0.13 | 0.38 |
| 10. | Propylparaben | 0.027 | 0.08 |
| 11. | Flavour | 0.01 | 0.03 |
| 12. | Colorant | 0.001 | 0.001 |
| | Total | | 300 |

### Example 18

| S. | | Quantity | Amount per |
|---|---|---|---|
| No | Ingredients | (% w/w) | strip in mg |
| 1. | Lactic acid (90%) | 37.29 | 220 |
| 2. | Fructooligosaccharides | 6.78 | 40 |
| 3. | Hydroxypropyl | 36.28 | 214.048 |
| | methylcellulose E50 | | |
| 4. | Propylene glycol | 3.39 | 20 |
| 5. | Glycerin | 3.39 | 20 |
| 6. | Maize starch | 4.24 | 25 |
| 7. | Silicon dioxide | 3.39 | 20 |
| 8. | Sodium hydroxide | 5.08 | 30 |
| 9. | Methylparaben | 0.13 | 0.75 |
| 10. | Propylparaben | 0.025 | 0.15 |
| 11. | Flavour | 0.008 | 0.05 |
| 12. | Colorant | 0.0003 | 0.002 |
| | Total | | 590 |

### Example 19

| S. | | Quantity | Amount per |
|---|---|---|---|
| No | Ingredients | (% w/w) | strip in mg |
| 1. | Lactic acid (90%) | 39.15 | 278 |
| 2. | Fructooligosaccharides | 7.04 | 50 |
| 3. | Hydroxypropyl | 32.87 | 233.368 |
| | methylcellulose E50 | | |
| 4. | Propylene glycol | 3.52 | 25 |
| 5. | Glycerin | 3.52 | 25 |
| 6. | Maize starch | 5.63 | 40 |
| 7. | Silicon dioxide | 2.82 | 20 |
| 8. | Sodium hydroxide | 5.28 | 37.5 |
| 9. | Methylparaben | 0.13 | 0.9 |
| 10. | Propylparaben | 0.025 | 0.18 |
| 11. | Flavour | 0.007 | 0.05 |
| 12. | Colorant | 0.0003 | 0.002 |
| | Total | | 710 |

### Example 20

| S. | | Quantity | Amount per |
|---|---|---|---|
| No | Ingredients | (% w/w) | strip in mg |
| 1. | Lactic acid | 34.27 | 220.00 |
| 2. | Sodium hydroxide | 4.67 | 30.00 |
| 3. | Fructooligosaccharides | 3.12 | 20.00 |
| 4. | Maize starch | 3.89 | 25.00 |
| 5. | Polyvinyl alcohol | 26.48 | 170.00 |
| 6. | Hydroxy propyl methyl | 18.69 | 120.00 |
| | cellulose E 15 | | |
| 7. | Glycerine | 1.56 | 10.00 |
| 8. | Methyl paraben | 0.12 | 0.75 |
| 9. | Propyl paraben | 0.02 | 0.15 |
| 10. | Silicon dioxide | 3.12 | 20.00 |
| 11. | Propylene glycol | 1.56 | 10.00 |
| 12. | Xanthan gum | 1.56 | 10.00 |
| 13. | Simethicone | 0.16 | 1.00 |
| 14. | Titanium dioxide | 0.08 | 0.50 |
| 15. | Tea tree oil | 0.71 | 4.55 |
| 16. | Approved colour | 0.01 | 0.05 |
| 17. | Purified water | 0.00 | 51.36 |
| | Total | 100.00 | 642.00 |

### Example 21

| S. | | Quantity | Amount per |
|---|---|---|---|
| No | Ingredients | (% w/w) | strip in mg |
| 1. | Lactic acid | 34.21 | 220.00 |
| 2. | Sodium hydroxide | 4.67 | 30.00 |
| 3. | Fructooligosaccharides | 3.11 | 20.00 |
| 4. | Maize starch | 3.89 | 25.00 |
| 5. | Polyvinyl alcohol | 26.44 | 170.00 |
| 6. | Hydroxy propyl methyl | 18.66 | 120.00 |
| | cellulose E 15 | | |
| 7. | Glycerine | 1.56 | 10.00 |
| 8. | Methyl paraben | 0.12 | 0.75 |
| 9. | Propyl paraben | 0.02 | 0.15 |
| 10. | Silicon dioxide | 3.11 | 20.00 |
| 11. | Propylene glycol | 1.56 | 10.00 |
| 12. | Xanthan gum | 1.56 | 10.00 |
| 13. | Simethicone | 0.31 | 2.00 |
| 14. | Titanium dioxide | 0.08 | 0.50 |
| 15. | Tea tree oil | 0.71 | 4.55 |
| 16. | Approved colour | 0.01 | 0.05 |
| 17. | Purified water | 0.00 | 51.36 |
| | Total | 100.00 | 643.00 |

### Example 22

| S. | | Quantity | Amount per |
|---|---|---|---|
| No | Ingredients | (% w/w) | strip in mg |
| 1. | Lactic acid | 34.29 | 220.00 |
| 2. | Sodium hydroxide | 4.68 | 30.00 |
| 3. | Fructooligosaccharides | 3.12 | 20.00 |
| 4. | Maize starch | 3.90 | 25.00 |
| 5. | Polyvinyl alcohol | 26.50 | 170.00 |
| 6. | Hydroxy propyl methyl | 18.71 | 120.00 |
| | cellulose E 15 | | |
| 7. | Glycerine | 1.56 | 10.00 |
| 8. | Methyl paraben | 0.12 | 0.75 |
| 9. | Propyl paraben | 0.02 | 0.15 |
| 10. | Silicon dioxide | 3.12 | 20.00 |
| 11. | Propylene glycol | 1.56 | 10.00 |
| 12. | Xanthan gum | 1.56 | 10.00 |
| 13. | Simethicone | 0.08 | 0.5 |
| 14. | Titanium dioxide | 0.08 | 0.50 |
| 15. | Tea tree oil | 0.71 | 4.55 |
| 16. | Approved colour | 0.01 | 0.05 |
| 17. | Purified water | 0.00 | 51.36 |
| | Total | 100.00 | 641.50 |

The vaginal film prepared as per the Example no. 20 of the present invention is evaluated for the stability at different conditions and the data is given below tables;

### Stability Studies:

| | | 40° C./75% RH | | | 30° C/75% RH | | 25° C./60% RH | |
|---|---|---|---|---|---|---|---|---|
| Tests | Initial | 1 M | 3 M | 6 M | 3 M | 6 M | 3 M | 6 M |
| Description | Pink to | Complies | Complies | Complies | Complies | Complies | Complies | Complies |
| | faintly | | | | | | | |
| | pink | | | | | | | |
| | coloured | | | | | | | |
| | square | | | | | | | |
| | shaped | | | | | | | |
| | film | | | | | | | |
| Assay (% w/w) | 111.9 | 109.6 | 107.9 | 110.2 | 109.2 | 106.8 | 112.6 | 110.6 |
| Disintegration | 1 min | 1 min | 1 min | 1 min | 1 min | 1 min | 1 min | 1 min |
| time | 30 sec | 46 sec | 36 sec | 34 sec | 48 sec | 42 sec | 29 sec | 31 sec |
| Water content | 7.01 | 5.96 | 7.50 | 6.76 | 6.38 | 6.47 | 6.88 | 7.31 |
| (% w/w) | | | | | | | | |
| pH (10% | 3.450 | 3.609 | 3.578 | 3.543 | 3.581 | 3.464 | 3.553 | 3.629 |
| dispersion) | | | | | | | | |
| Folding | 275 | 298 | 237 | 276 | 291 | 265 | 275 | 269 |
| endurance (No | | | | | | | | |
| of folds) | | | | | | | | |

1. An organic acid vaginal film composition comprising Lactic acid as active ingredient, optionally prebiotics, film forming agents, plasticizers, neutralizing agent, lubricant as pharmaceutically acceptable excipients.

2. The organic acid vaginal film composition as claimed in claim 1, further contains optional excipients selected from preservatives, disintegrating agent, anti-foaming agent, antimicrobial agent, coloring agent and flavouring agent.

3. The organic acid vaginal film composition as claimed in claim 1, wherein the Lactic acid is in the range of 10% to 45% (w/w), preferably 23.28% to 39.29% (w/w) of the total weight of the composition.

4. The organic acid vaginal film composition as claimed in claim 1, wherein prebiotics is selected from inulin, fructooligosaccharides (FOS), galactooligosaccharides (GOS), lactulose, polydextrose, isomaltooligosaccharides (IMO), xylooligosaccahrides (XOS), lactitol, chicory root inulin-derived, wheat bran-derived arabinoxylooligosaccharides (AXOS), xylooligosaccharides (XOS) and prebiotics can also be found in some vegetables, such as leeks, onions, chicory, tomatoes, asparagus, artichokes, bananas, alfalfa.

5. The organic acid vaginal film composition as claimed in claim 4, wherein the prebiotics is in the range of 3% to 10% (w/w), preferably 3.11% to 7.14% (w/w) of the total weight of the composition.

6. The organic acid vaginal film composition as claimed in claim 1, wherein the film forming agents are selected from hydroxyethyl cellulose, carrageen, eudragit EPO, hydroxypropyl cellulose, hydroxypropyl methylcellulose, gums, starch, polymerized rosin, pullulan, sodium alginate, pectin, gelatine, chitosan, maltodextrins, polyvinyl alcohol, sodium carboxy methyl cellulose, copovidone, polyvinyl pyrrolidone, poloxamer, ammonium alginate, ethyl cellulose, ethyl lactate, hydroxy propyl methyl cellulose acetate succinate, poly ethylene oxide, higher molecular weight poly ethylene glycols, poly methacrylates, poly (methyl vinyl ether/maleic anhydride, polyvinyl acetate phthalate and shellac.

7. The organic acid vaginal film composition as claimed in claim 6, wherein the film forming agent is in the range of 1% to 40% (w/w), preferably used concentration of film forming agent individually is from 1.56% to 37.84%, preferably used concentration of film forming agent in combination is from 15% to 50% (w/w) of the total weight of the composition.

8. The organic acid vaginal film composition as claimed in claim 1, wherein the plasticizers are selected from triacetin, low molecular weight polyethylene glycols, triethyl citrate, glycerine, propylene glycol, acetyltributyl citrate, acetyltriethyl citrate, benzyl benzoate, cellulose acetate phthalate compatible, chlorbutanol, dextrin, dibutyl phthalate, dibutyl sebacate, diethyl phthalate, dimethyl phthalate, glycerin monostearate, mannitol, mineral oil and lanolin alcohols, palmitic acid, petrolatum and lanolin alcohols, pyrrolidone, sorbitol, tributyl citrate, triethanolamine and vitamin E TPGS.

9. The organic acid vaginal film composition as claimed in claim 8, wherein the plasticizers is in the range of 1% to 40% (w/w), preferably used concentration of plasticizers individually is from 1.08% to 37.04% (w/w), preferably used concentration of plasticizers in combination is from 2% to 20% (w/w).

10. The organic acid vaginal film composition as claimed in claim 1, wherein the neutralizing agent is selected from sodium carbonate, sodium bicarbonate, potassium carbonate, potassium bicarbonate, sodium phosphate dibasic, sodium phosphate tribasic, potassium phosphate dibasic, potassium phosphate tribasic, calcium carbonate, magnesium carbonate, sodium hydroxide, magnesium hydroxide, potassium hydroxide, aluminum hydroxide and combinations thereof.

11. The organic acid vaginal film composition as claimed in claim 10, wherein the neutralizing agent is in the range of 2% to 8% (w/w), preferably 3.23% to 5.36% (w/w) of the total weight of the composition.

12. The organic acid vaginal film composition as claimed in claim 1, wherein the lubricant is selected from stearic acid, calcium stearate, glycerin monostearate, glyceryl behenate, glyceryl palmitostearate, hydrogenated castor oil, light mineral oil, magnesium lauryl sulfate, magnesium stearate, medium-chain triglycerides, mineral oil, myristic acid, palmitic acid, sodium benzoate, sodium lauryl sulfate, sodium stearyl fumarate, talc, zinc stearate and silicon dioxide.

13. The organic acid vaginal film composition as claimed in claim 12, wherein the lubricant is in the range of 1% to 20% (w/w), preferably 2.82% to 18.75% (w/w) of the total weight of the composition.

14. The organic acid vaginal film composition as claimed in claim 2, wherein the preservatives are selected from alkanols, disodium EDTA (ethylenediamine tetraacetate), EDTA salts, EDTA fatty acid conjugates, isothiazolinone, benzoic esters (parabens) (e.g., methylparaben, propylparaben, butylparaben, ethylparaben, isopropylparaben, isobutylparaben, benzylparaben, sodium methylparaben, and sodium propylparaben), benzoic acid, propylene glycols, sorbates and urea derivatives (e.g., diazolindinyl urea).

15. The organic acid vaginal film composition as claimed in claim 14, wherein the preservatives is in the range of 0.01% to 0.2% (w/w), preferably used concentration of preservatives individually is from 0.02% to 0.13% (w/w), preferably used concentration of preservatives in combination is from 0.14 to 0.16% (w/w) of the total weight of the composition.

16. The organic acid vaginal film composition as claimed in claim 2, wherein the disintegrating agent is starch, lactose, monohydrate, corn starch, alginic acid, calcium alginate, cellulose, powdered, croscarmellose sodium, crospovidone, docusate sodium, glycine, magnesium aluminum silicate, methylcellulose, microcrystalline cellulose, polacrilin potassium, sodium alginate and sodium starch glycolate.

17. The organic acid vaginal film composition as claimed in claim 16, wherein the disintegrating agent is in the range of 3% to 7% (w/w), preferably 3.89% to 5.63% (w/w) of the total weight of the composition.

18. The organic acid vaginal film composition as claimed in claim 2, wherein the anti foaming agent is selected from simethicone, alcohols like cetostearyl alcohol, insoluble oils (castor oil), oleic acid, stearates, other silicones derivatives, ether, glycols, 2-octanol, paraffinic waxes, amide waxes, sulfonated oils, organic phosphates, silicone oils and mineral oils.

19. The organic acid vaginal film composition as claimed in claim 18, wherein the anti-foaming agent is in the range of 0.05% to 0.5% (w/w), preferably 0.08% to 0.31% (w/w) of the total weight of the composition.

20. The organic acid vaginal film composition as claimed in claim 2, wherein the antimicrobial agent is selected from oregano oil, basil oil, rosemarin oil, eucalyptus oil, tea tree oil or thyme oil.

21. The organic acid vaginal film composition as claimed in claim 20, wherein the antimicrobial agent is in the range of 0.5% to 1% (w/w), preferably 0.71% (w/w) of the total weight of the composition.

22. The organic acid vaginal film composition as claimed in claim 2, wherein the coloring agent is selected from titanium dioxide, FD&C, D&C, lakes, approved colours etc.

23. The organic acid vaginal film composition as claimed in claim 22, wherein the coloring agent is in the range of 0.0001% to 0.6% (w/w), preferably 0.0003% to 0.53% (w/w) of the total weight of the composition.

24. The organic acid vaginal film composition as claimed in claim 2, wherein the flavouring agent includes any acceptable flavour and in the range of 0.005% to 0.03% (w/w), preferably 0.007% to 0.01% (w/w) of the total weight of the composition.

25. A process for preparing vaginal film composition comprising Lactic acid as active ingredient, optionally prebiotics, film forming agents, plasticizers, neutralizing agent, lubricant as pharmaceutically acceptable excipients, wherein the process comprising steps of:
(a) dissolving active ingredient and neutralizing agent in solvent under continuous stirring and adding optionally prebiotics, coloring agents, flavouring agent, anti-foaming agent, antimicrobial agent and preservatives,
(b) melting one or more lubricants, plasticizers and film forming agents under heating and continuous stirring and adding disintegrating agents,
(c) adding step (a) to step (b) under continuous heating, stirring and mixing it until uniform mixture is formed,
(d) coating the above solution on suitable liner like polyethylene terephthalate by using hot melt coater at 80 to 140° C.,
(e) cooling down the coat to room temperature, and
(f) cutting into desired size to get vaginal film, packing and sealing the film into sachets.

26. A process for preparing vaginal film composition comprising Lactic acid as active ingredient, optionally prebiotics, film forming agents, plasticizers, neutralizing agent, lubricant as pharmaceutically acceptable excipients, wherein the process comprising steps of:
(a) dissolving active ingredient and neutralizing agent in solvent under continuous stirring,
(b) adding one or more lubricants, disintegrating agents, plasticizers, coloring agents, flavouring agent and preservatives to step (a) under continuous stirring,
(c) adding film forming agent to step (b) under continuous stirring and mixing it until uniform mixture is formed,
(d) coating the above solution on suitable liner like polyethylene terephthalate by using film applicator,
(e) drying the coated liquid polymeric film in hot air oven, and
(f) cutting into desired size to get vaginal film, packing and sealing the film into sachets.

27. A process for preparing vaginal film composition comprising Lactic acid as active ingredient, optionally prebiotics, film forming agents, plasticizers, neutralizing agent, lubricant as pharmaceutically acceptable excipients, wherein the process comprising steps of:
(a) dissolving active ingredient and neutralizing agent in solvent under continuous stirring,
(b) adding one or more prebiotics, lubricants, disintegrating agents, plasticizers, coloring agents, flavouring agent and preservatives to step (a) under continuous stirring,
(c) dispersing film forming agent in solvent in a separate vessel under continuous stirring and cooling it,
(d) adding step (b) into step (c) with continuous stirring until uniform mixture is formed,
(e) coating the above solution on suitable liner like polyethylene terephthalate by using film applicator,
(f) drying the coated liquid polymeric film in hot air oven, and
(g) cutting into desired size to get vaginal film, packing and sealing the film into sachets.

28. The process for preparing vaginal film as claimed in claim 25, wherein the process comprising steps of:
(a) dissolving Lactic acid and sodium hydroxide in demineralized water under continuous stirring and adding optionally fructooligosaccharides, coloring agents, flavouring agent, simethicone, tea tree oil, methylparaben and propylparaben,
(b) melting stearic acid and/or silicon dioxide, polyethylene glycol or triethyl citrate or vitamin E TPGS or glycerine and eudragit or gelatine or copovidone or polyvinyl pyrrolidine or hydroxy propyl cellulose or poloxamer under heating and continuous stirring and adding starch,
(c) adding step (a) to step (b) under continuous heating, stirring and mixing it until uniform mixture is formed,
(d) coating the above solution on suitable liner like polyethylene terephthalate by using hot melt coater at 80 to 140° C.,
(e) cooling down the coat to room temperature, and
(f) cutting into desired size to get vaginal film, packing and sealing the film into sachets.

29. The process for preparing vaginal film as claimed in claim 26, wherein the process comprising steps of:
(a) dissolving Lactic acid and sodium hydroxide in demineralized water under continuous stirring,
(b) adding silicon dioxide, starch, propylene glycol or glycerin, colorant, flavor, methylparaben and propylparaben to step (a) under continuous stirring,
(c) adding polyvinyl alcohol or hydroxypropyl methylcellulose to step (b) under continuous stirring and mixing it until uniform mixture is formed,
(d) coating the above solution on suitable liner like polyethylene terephthalate by using film applicator,
(e) drying the coated liquid polymeric film in hot air oven at 50 to 80° C. for 45 to 180 minutes, and
(f) cutting into desired size to get vaginal film, packing and sealing the film into sachets.

30. The process for preparing vaginal film as claimed in claim 27, wherein the process comprising steps of:
(a) dissolving Lactic acid and sodium hydroxide in demineralized water under continuous stirring,
(b) adding fructooligosaccharides, silicon dioxide, starch, propylene glycol or glycerin, colorant, flavor, methylparaben and propylparaben to step (a) under continuous stirring,
(c) dispersing polyvinyl alcohol or hydroxypropyl methylcellulose into purified water at 70° C. in a separate vessel under continuous stirring and cooling to 50° C.,
(d) adding step (b) into step (c) with continuous stirring until uniform mixture is formed,
(e) coating the above solution on suitable liner like polyethylene terephthalate by using film applicator,
(f) drying the coated liquid polymeric film at average temperature of 70 to 80° C. for 30 to 90 minutes, and
(g) cutting into desired size to get vaginal film, packing and sealing the film into sachets.
